(19) United States
(12) Patent Application Publication
(10) Pub. No.: US 2025/0049416 A1
(43) Pub. Date: Feb. 13, 2025

(54) ROBOT DEVICE

(71) Applicant: FUJI CORPORATION, Chiryu (JP)

(72) Inventor: Keisuke NAGIRI, Nagoya-shi (JP)

(73) Assignee: FUJI CORPORATION, Chiryu (JP)

(21) Appl. No.: 18/844,455

(22) PCT Filed: Apr. 8, 2022
(86) PCT No.: PCT/JP2022/017336

§ 371 Date: Sep. 6, 2024

(51) Int. Cl. A61B 8/00; A61B 34/30; A61B 46/10; A61B 90/30

(52) U.S. Cl. CPC A61B 8/4218; A61B 8/4405; A61B 34/30; A61B 46/10; A61B 90/30

(57) ABSTRACT

A robot device is used in an ultrasound diagnostic system configured to perform an ultrasound diagnosis on a subject. The robot device includes an arm configured to hold and move an ultrasound probe, and an irradiation unit configured to irradiate the subject with light indicating a moving range of the ultrasound probe.

### TECHNICAL FIELD

The present description discloses a robot device.

### BACKGROUND ART

Conventionally, there is known a robot device that irradiates a subject with ultrasound while changing the posture of an ultrasound probe using a robot arm, thereby performing an ultrasound diagnosis on the subject. For example, Patent Literature 1 discloses a robot device that displays an irradiation range of ultrasound on a monitor.

### PATENT LITERATURE

Patent Literature 1: JP-A-2018-42900

### BRIEF SUMMARY

### Technical Problem

In such a robot device, an operator may operate the robot arm in order to register the movement trajectory of the ultrasound probe. Such operations are performed as follows, for example. That is, the operator sets the robot device such that the subject is included within a moving range of the ultrasound probe. Then, the operator registers the movement trajectory of the ultrasound probe so that an ultrasound diagnosis of the subject can be performed. When a series of operations are performed, the operator often needs to operate the robot arm after obtaining the moving range of the ultrasound probe based on sensory perception. Although it is conceivable to display the moving range of the ultrasound probe on the monitor, the operator needs to visually check the monitor to confirm the moving range, and cannot operate the robot arm while maintaining constant visual contact with the subject. Thus, it is desired to improve work efficiency when the operator operates the robot arm.

An object of the present disclosure is to improve work efficiency when an operator operates an arm.

### Solution to Problem

A robot device according to an embodiment of the present disclosure is a robot device used in an ultrasound diagnostic system configured to perform an ultrasound diagnosis on a subject, including:
an arm configured to hold and move an ultrasound probe; and
an irradiation unit configured to irradiate the subsect with light indicating a moving range of the ultrasound probe.

In this robot device, the irradiation unit irradiates the subject with light indicating the moving range of the ultrasound probe. As a result, an operator can obtain the moving range of the ultrasound probe without relying on sensory perception and operate the arm while maintaining constant visual contact with the subject. Accordingly, the work efficiency when the operator operates the arm improves.

### BRIEF DESCRIPTION OF DRAWINGS

FIG. 1 is a configuration diagram illustrating an outline of a configuration of ultrasound diagnostic system 10.

FIG. 2 is a front view of robot device 20.

FIG. 3 is a side view of robot device 20.

FIG. 4 is a diagram illustrating an example of an ultrasound diagnostic procedure.

FIG. 5 is a view illustrating a state in which irradiation units 61a and 61b emit light La and Lb.

### DESCRIPTION OF EMBODIMENTS

Next, an embodiment of the present disclosure will be described with reference to the drawings.

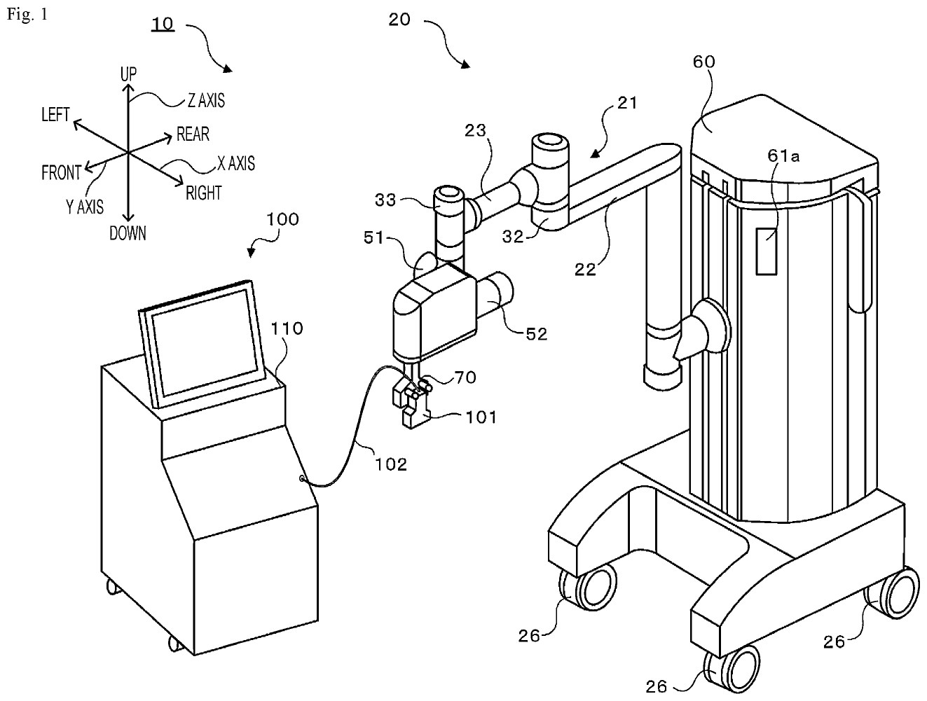

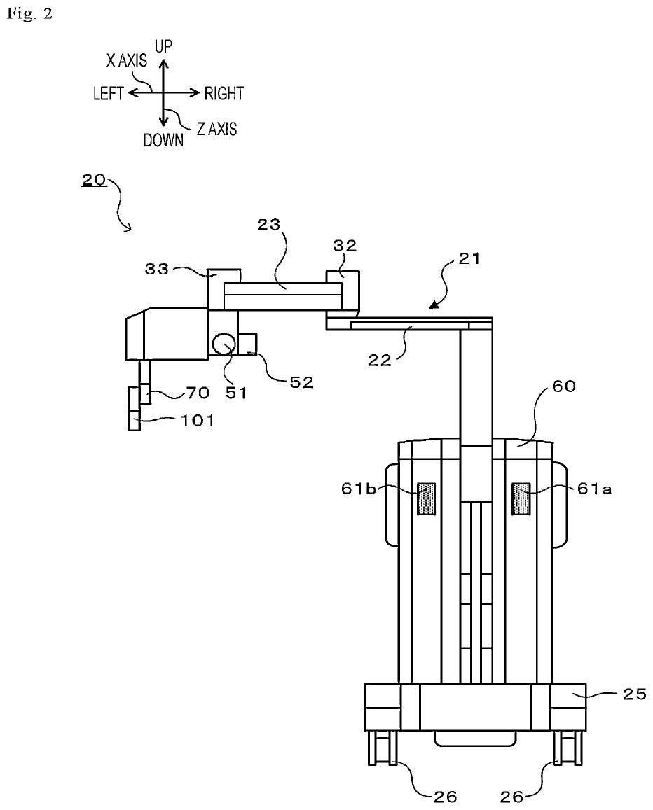

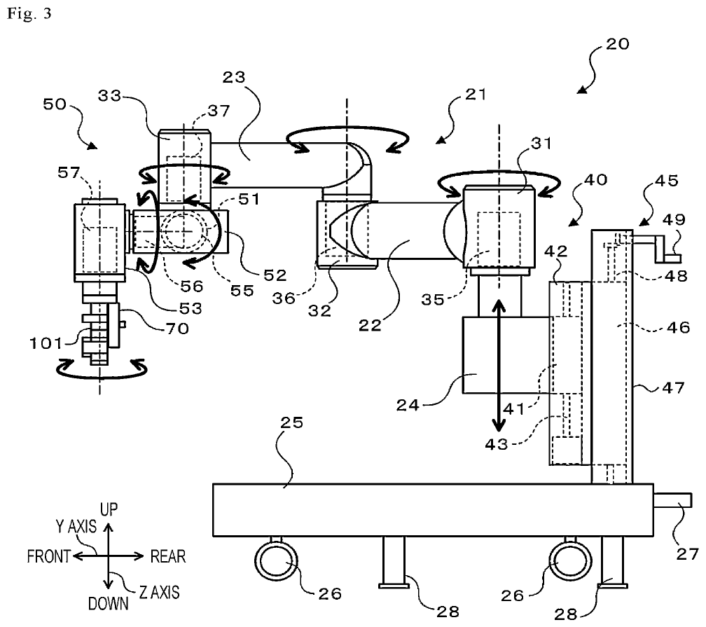

FIG. 1 is a configuration diagram illustrating an outline of a configuration of ultrasound diagnostic system 10. FIG. 2 is a front view of robot device 20. FIG. 3 is a side view of robot device 20. In FIG. 3, illustration of cover 60 is omitted. In FIGS. 1 to 3, the left-right direction is an X-axis direction (vertical direction on the page in FIG. 3), the front-rear direction is a Y-axis direction (vertical direction on the page in FIG. 2), and the up-down direction is a Z-axis direction.

Ultrasound diagnostic system 10 of the present embodiment obtains an ultrasound echo image by holding ultrasound probe 101 on a hand of robot arm 21 and operating robot device 20 such that ultrasound probe 101 is pressed against a body surface of patient P. Ultrasound diagnostic system 10 is used in, for example, catheter treatment. An operator (technician) who operates a guide wire of a catheter can press ultrasound probe 101 against a body surface of patient P and moves the guide wire forward while recognizing a positional relationship between a front end of the guide wire and a blood vessel from the obtained ultrasound echo image, thereby causing the guide wire to accurately pass through the center of an occluded portion or a stenosed portion of the blood vessel.

Ultrasound diagnostic system 10 includes ultrasound diagnostic device 100 and robot device 20.

As illustrated in FIG. 1, ultrasound diagnostic device 100 includes ultrasound probe 101 and ultrasound diagnostic device main body 110 coupled to ultrasound probe 101 through cable 102. Ultrasound diagnostic device main body 110 includes an ultrasound diagnostic control section that controls the entire device, an image processing section that processes a reception signal from ultrasound probe 101 to generate an ultrasound echo image, and an image display section that displays the ultrasound echo image.

As illustrated in FIGS. 1 to 3, robot device 20 is a medical robot including base plate 25, robot arm 21 installed on base plate 25, height adjustment mechanism 45 that adjusts a height of robot arm 21 by a manual operation, cover 60, and irradiation units 61a and 61b. Further, robot device 20 also includes a control device (not illustrated) and the like.

As illustrated in FIGS. 1 to 3, casters 26 respectively having stoppers are attached to four corners of a rear surface of base plate 25. Robot device 20 can be moved more freely by casters 26. Further, at multiple locations (for example, three locations) on the rear surface of base plate 25, lock sections 28 are provided which protrude vertically downward by pushing down lever 27 and lock (fix) robot device 20 so as not to be movable.

As illustrated in FIG. 3, robot arm 21 includes first arm 22, first arm drive device 35, second arm 23, second arm drive device 36, base 24, rotation three-axis mechanism 50, and posture holding device 37.

A base end portion of first arm 22 is coupled to base 24 through first joint shaft 31 extending in an up-down direction (the Z-axis direction). First arm drive device 35 includes a motor, an encoder, and an amplifier. A rotation shaft of the motor is coupled to first joint shaft 31 through a decelerator (not illustrated). First arm drive device 35 causes first arm 22 to rotate (revolve) along a horizontal plane (XY plane) around first joint shaft 31 as a fulcrum by rotationally driving first joint shaft 31 by the motor. The encoder is attached to the rotation shaft of the motor and is configured as a rotary encoder that detects a rotational displacement amount of the motor. The amplifier is a drive section for driving the motor by switching of a switching element.

A base end portion of second arm 23 is coupled to a front end portion of first arm 22 through second joint shaft 32 extending in the up-down direction. Second arm drive device 36 includes a motor, an encoder, and an amplifier. A rotation shaft of the motor is coupled to second joint shaft 32 through a decelerator (not illustrated). Second arm drive device 36 causes second arm 23 to rotate (revolve) along a horizontal plane around second joint shaft 32 as a fulcrum by rotationally driving second joint shaft 32 by the motor. The encoder is attached to the rotation shaft of the motor and is configured as a rotary encoder that detects a rotational displacement amount of the motor. The amplifier is a drive section for driving the motor by switching of a switching element.

Base 24 is provided to be lifted and lowered with respect to base plate 25 by lifting and lowering device 40 installed on base plate 25. As illustrated in FIG. 3, lifting and lowering device 40 includes first slider 41 fixed to base 24, first guide member 42 extending in the up-down direction and guiding the movement of first slider 41, first ball screw shaft 43 (lifting and lowering shaft) extending in the up-down direction and screwed into a ball screw nut (not illustrated) fixed to first slider 41, a motor for rotationally driving first ball screw shaft 43, an encoder, and an amplifier for driving the motor. Lifting and lowering device 40 moves base 24 fixed to first slider 41 in the up-down direction along first guide member 42 by rotationally driving first ball screw shaft 43 by the motor. The encoder is configured as a linear encoder that detects a position (lifting and lowering position) of first slider 41 (base 24) in the up-down direction.

As illustrated in FIG. 3, rotation three-axis mechanism 50 is coupled to a front end portion of second arm 23 through posture holding shaft 33 extending in the up-down direction. Rotation three-axis mechanism 50) includes first rotation shaft 51, second rotation shaft 52, and third rotation shaft 53 that are orthogonal to each other, first rotation device 55 that rotates first rotation shaft 51, second rotation device 56 that rotates second rotation shaft 52, and third rotation device 57 that rotates third rotation shaft 53. First rotation shaft 51 is supported in a posture orthogonal to posture holding shaft 33. Second rotation shaft 52 is supported in a posture orthogonal to first rotation shaft 51. Third rotation shaft 53 is supported in a posture orthogonal to second rotation shaft 52. First rotation device 55 includes a motor that rotationally drives first rotation shaft 51, an encoder that is attached to a rotation shaft of the motor and detects a rotational displacement amount of the motor, and an amplifier that drives the motor. Second rotation device 56 includes a motor that rotationally drives second rotation shaft 52, an encoder that is attached to a rotation shaft of the motor and detects a rotational displacement amount of the motor, and an amplifier that drives the motor. Third rotation device 57 includes a motor that rotationally drives third rotation shaft 53, an encoder that is attached to a rotation shaft of the motor and detects a rotational displacement amount of the motor, and an amplifier that drives the motor. Further, third rotation shaft 53 includes holding section 70 for holding ultrasound probe 101. Holding section 70 holds ultrasound probe 101 to be located coaxially with third rotation shaft 53.

Posture holding device 37 holds the posture (orientation of first rotation shaft 51) of rotation three-axis mechanism 50 in a constant orientation regardless of postures of first arm 22 and second arm 23. Posture holding device 37 includes a motor, an encoder, and an amplifier. A rotation shaft of the motor is coupled to posture holding shaft 33 through a decelerator (not illustrated). Posture holding device 37 sets a target rotation angle of posture holding shaft 33 based on a rotation angle of first joint shaft 31 and a rotation angle of second joint shaft 32 such that an axial direction of first rotation shaft 51 is constantly in the left-right direction (X-axis direction), and drives and controls the motor such that posture holding shaft 33 is at the target rotation angle. Thereby, a translational motion in three directions and a rotational motion in three directions can be independently controlled, and thus, control is easily made.

Within the movable range, robot device 20 of the present embodiment can move ultrasound probe 101 to a certain position in any posture by combining a translational motion in three directions of the X-axis direction, the Y-axis direction, and the Z-axis direction by first arm drive device 35, second arm drive device 36, and lifting and lowering device 40 with a rotational motion in three directions of an X-axis spin (pitching), a Y-axis spin (rolling), and a Z-axis spin (yawing) by rotation three-axis mechanism 50.

As illustrated in FIG. 3, height adjustment mechanism 45 includes second slider 46 fixed to first guide member 42 of lifting and lowering device 40, second guide member 47 fixed to base plate 25 and extending in the up-down direction to guide the movement of second slider 46, second ball screw shaft 48 (lifting and lowering shaft) extending in the up-down direction and screwed into a ball screw nut (not illustrated) fixed to second slider 46, and operation handle 49 coupled to second ball screw shaft 48 through a power transfer mechanism (bevel gear). Height adjustment mechanism 45 moves first guide member 42 of lifting and lowering device 40 fixed to second slider 46 up and down along second guide member 47 by rotationally driving second ball screw shaft 48 by a manual operation of operation handle 49. A base end of robot arm 21 is fixed to base 24, and base 24 is supported by first guide member 42, and accordingly, a height of robot arm 21 can be adjusted by moving first guide member 42 up and down by using height adjustment mechanism 45. Thereby, the height of robot arm 21 can be adjusted according to a height of bed B on which, for example, patient P undergoing ultrasound diagnosis lies.

Cover 60 is a cover member that covers lifting and lowering device 40. As illustrated in FIGS. 1 and 2, cover 60 is fixed to base plate 25. Cover 60 is disposed in front of lifting and lowering device 40 and height adjustment mechanism 45.

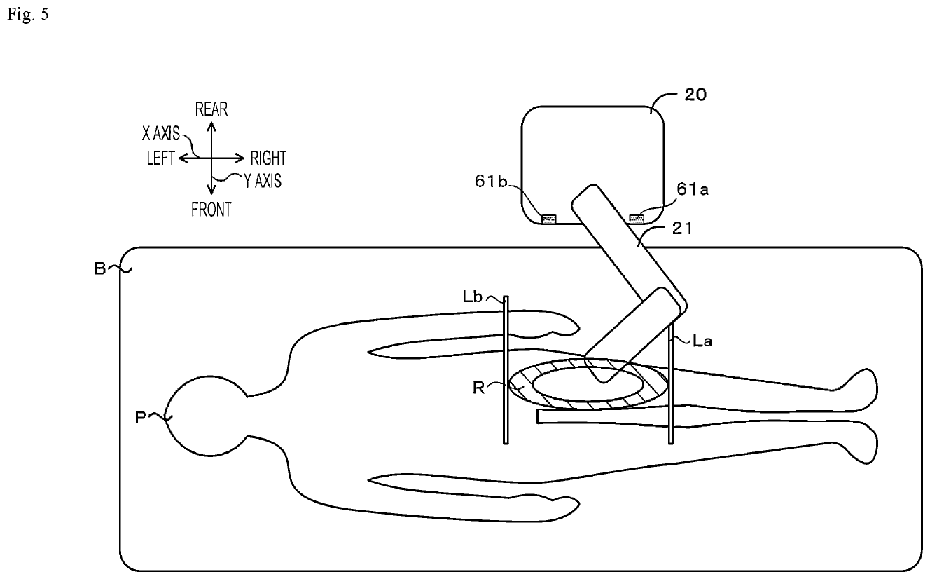

Irradiation units 61a and 61b are units that irradiate the body surface of patient P with light La and Lb (see FIG. 5), which indicates the boundaries of moving range R of ultrasound probe 101 held by robot arm 21. Here, moving range R is set to be slightly narrower than the movable range of ultrasound probe 101. Light La and Lb is linear light and indicates the boundaries in the left-right direction of moving range R. Irradiation units 61a and 61b are provided on the front surface of cover 60. As illustrated in FIG. 2, irradiation unit 61a is installed on the right side of robot arm 21 when cover 60 is viewed from the front. As illustrated in FIG. 2, irradiation unit 61b is installed on the left side of robot arm 21 when cover 60 is viewed from the front. Irradiation units 61a and 61b are configured, for example, as laser modules or LED modules.

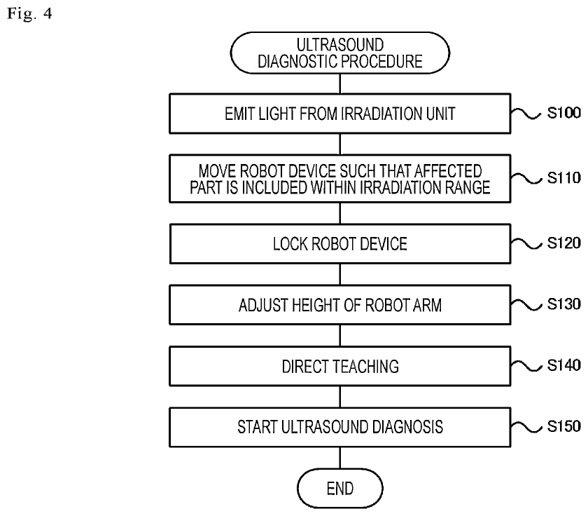

Next, a usage example of robot device 20 configured in this way will be described with reference to FIGS. 4 and 5. FIG. 4 is a diagram illustrating an example of an ultrasound diagnostic procedure. FIG. 5 is a view illustrating a state in which irradiation units 61a and 61b emit light La and Lb. This procedure is performed by the operator. When the operator performs this procedure, patient P is lying on bed B.

When this procedure is started, the operator operates a switch (not illustrated) to emit light La and Lb from irradiation units 61a and 61b (step S100). Next, as illustrated in FIG. 5, the operator moves robot device 20 such that the affected part of patient P is included between light La and light Lb (step S110).

Subsequently, the operator locks robot device 20 (step S120). Specifically, the operator locks the stoppers of casters 26. Then, the operator pushes down lever 27. Thus, lock section 28 protrudes vertically downward from base plate 25 to lock robot device 20 so as not to be movable.

Next, the operator adjusts the height of robot arm 21 (step S130). Specifically, the operator rotates operation handle 49 to adjust robot arm 21 to a height corresponding to the height of bed B.

Next, the operator performs direct teaching (step S140). The direct teaching is an operation in which the operator manually operates robot arm 21 to register the movement trajectory of ultrasound probe 101 into the control device of robot device 20. At this time, irradiation units 61a and 61b irradiate the body surface of patient P with light La and Lb, as boundaries of moving range R of ultrasound probe 101. As a result, during direct teaching, the operator can obtain the boundaries of moving range R of ultrasound probe 101 without relying on sensory perception and operate robot arm 21 while maintaining constant visual contact with patient P.

Then, the operator starts ultrasound diagnosis (step S150). When the ultrasound diagnosis is started, the control device of robot device 20 controls various members so that ultrasound probe 101 moves according to the movement trajectory registered in S150 in accordance with an instruction input from the operator. The image processing section of ultrasound diagnostic device main body 110 processes a reception signal from ultrasound probe 101 to generate an ultrasound echo image. The ultrasound diagnostic control section of ultrasound diagnostic device main body 110 causes the image display section to display the ultrasound echo image. The operator diagnoses patient P based on the echo image displayed on the image display section. After step S150, the operator ends this procedure.

Here, a correspondence relationship between main elements of the embodiment and main elements of the present disclosure described in the claims will be described. That is, robot device 20 of the present embodiment corresponds to the robot device of the present disclosure, where robot arm 21 corresponds to the arm, irradiation units 61a and 61b correspond to the irradiation units, base 24 corresponds to the base, and cover 60 corresponds to the cover. Patient P corresponds to a subject.

In robot device 20 described above, irradiation units 61a and 61b irradiate patient P with light indicating moving range R of ultrasound probe 101. As a result, the operator can obtain moving range R of ultrasound probe 101 without relying on sensory perception and operate robot arm 21 while maintaining constant visual contact with patient P. Accordingly, the work efficiency when the operator operates robot arm 21 improves.

Robot device 20 is a medical robot that performs operations on patient P. and irradiation units 61a and 61b emit light La and Lb such that the affected part of patient P is included within moving range R of ultrasound probe 101. Therefore, the operator can easily set robot device 20 such that the affected part of patient P is included within moving range R of ultrasound probe 101, and is particularly useful.

Robot device 20 includes base 24 that supports robot arm 21 and cover 60 that covers base 24, with irradiation units 61a and 61b provided on cover 60. Therefore, the light emitted from irradiation units 61a and 61b is less likely to overlap with robot arm 21.

In robot device 20, irradiation units 61a and 61b are installed on the left and right sides of the front surface of cover 60 with robot arm 21 interposed therebetween. Therefore, two boundaries of moving range R of ultrasound probe 101 can be displayed.

Irradiation units 61a and 61b on both left and right sides irradiate the body surface of patient P with linear light La and Lb, as boundaries of moving range R. In this case, since two beams of linear light can represent the boundaries of moving range R, the operator can more easily obtain moving range R of ultrasound probe 101.

It is needless to say that the present disclosure is not limited in any way to the above-described embodiment, and the present disclosure can be embodied in various aspects as long as the aspects fall within the technical scope of the present disclosure.

For example, in the above-described embodiment, robot device 20 is configured as a seven-axes articulated robot capable of performing a translational motion in three directions and a rotational motion in three directions. However, the number of axes may be any number. Further, robot device 20 may be configured with a so-called vertical articulated robot, a horizontal articulated robot, or the like.

In the above-described embodiment, robot device 20 includes irradiation units 61a and 61b. However, robot device 20 may have only one of irradiation unit 61a or irradiation unit 61b. Alternatively, robot device 20 may have one or two other irradiation units that emit linear light in a direction orthogonal to light La and Lb. The light emitted by the other irradiation unit(s) indicates the boundaries of the front-rear direction within moving range R. In this case, the other irradiation unit(s) may be held by an irradiation unit holding member provided in front of robot arm 21. The irradiation unit holding member is provided separately from robot arm 21 and cover 60, for example.

In the above-described embodiment, irradiation units 61a and 61b irradiate two beams of linear light that are parallel to each other. However, the body surface of patient P may be irradiated with a rectangular or elliptical light corresponding to moving range R of ultrasound probe 101 using multiple laser modules and multiple LED modules.

### INDUSTRIAL APPLICABILITY

The present disclosure is applicable to an ultrasound diagnostic device, a manufacturing industry of a robot, and the like.

### REFERENCE SIGNS LIST

10: ultrasound diagnostic system, 20: robot device, 21: robot arm, 22: first arm, 23: second arm, 24: base, 25: base plate, 26: caster, 27: lever, 28: lock section, 31: first joint shaft, 32: second joint shaft, 33: posture holding shaft, 35: first arm drive device, 36: second arm drive device, 37: posture holding device, 40: lifting and lowering device, 41: first slider, 42: first guide member, 43: first ball screw shaft, 45: height adjustment mechanism, 46: second slider, 47: second guide member, 48: second ball screw shaft, 49: operation handle, 50: rotation three-axis mechanism, 51: first rotation shaft. 52: second rotation shaft, 53: third rotation shaft, 55: first rotation device, 56: second rotation device, 57: third rotation device, 60: cover, 61a, 61b: irradiation unit, 70: holding section, 100: ultrasound diagnostic device, 101: ultrasound probe, 102: cable, 110: ultrasound diagnostic device main body, B: bed, La, Lb: light. P: patient. R: moving range

1. A robot device used in an ultrasound diagnostic system configured to perform an ultrasound diagnosis on a subject, comprising:
an arm configured to hold and move an ultrasound probe; and
an irradiation unit configured to irradiate the subject with light indicating a moving range of the ultrasound probe.

2. The robot device according to claim 1,
wherein the robot device is a medical robot configured to perform operations on a patient,
the subject is the patient, and
the irradiation unit is configured to emit light such that an affected part of the patient is included within the moving range of the ultrasound probe.

3. The robot device according to claim 1, further comprising:
a base that supports the arm; and
a cover that covers the base,
wherein the irradiation unit is provided on the cover.

4. The robot device according to claim 3,
wherein the irradiation unit is installed on both left and right sides of a front surface of the cover with the arm interposed therebetween.

5. The robot device according to claim 4,
wherein the irradiation units on both left and right sides are configured to irradiate a surface of the subject with linear light, as boundaries of the moving range.
